(19) United States
(12) Patent Application Publication
(10) Pub. No.: US 2005/0090801 A1
(43) Pub. Date: Apr. 28, 2005

(54) Safety spinal catheter

(76) Inventors: N. Sandor Racz, Irving, TX (US); Christopher Wiley, Hanover, NH (US)

Correspondence Address: TRASK BRITT, P.O. BOX 2550, SALT LAKE CITY, UT, 84110, US

(21) Appl. No.: 10/694,235

(22) Filed: Oct. 27, 2003

(51) Int. Cl. A61M 31/00

(52) U.S. Cl. 604/500; 604/164.07

(57) ABSTRACT

An apparatus and method to insert a spinal catheter and minimize incidence of post dural puncture headache. The apparatus typically includes a support needle with a non-cutting piercing tip and an exteriorly mounted catheter. The catheter gauge is reduced, while ease of use is increased by the exterior mounting. The catheter may include a kink sleeve that protects against kinking when bent during use. A central stylet maybe included to prevent entry of matter into the support needle opening during insertion. Methods using a single stick insertion procedure allow for catheter placement to be determined and adjusted based on physical feedback obtained during the single stick insertion.

### TECHNICAL FIELD

This invention relates to medical catheters. It is particularly directed to spinal catheters.

### BACKGROUND

The advantages of continuous spinal anesthesia have long been appreciated by anesthesiologists. Unlike conventional single-shot techniques, continuous spinal anesthesia (“CSA”) with an indwelling catheter allows anesthesia of unlimited duration and the ability to carefully control the level of the block by repeated small incremental doses of anesthetic. As compared to continuous epidural anesthesia, which has become widely used as a substitute for spinal, CSA generally requires far less drug to achieve the desired effect, has a definite endpoint of correct catheter placement, requires no “test dose,” and produces a much more reliable, less spotty block.

Unfortunately, technical problems have severely limited the usefulness of continuous spinal techniques. Until recently, the standard technique of inserting the spinal catheter through the spinal needle, coupled with the difficulty of manufacturing truly small needles and catheters, has meant large needles and catheters were required, resulting in an unacceptably high incidence of post-dural puncture headache (“PDPH”).

In the mid 1980's, various advances fueled renewed interest in spinal anesthesia in general and in CSA in particular. Improvements in manufacturing ever-smaller conventional (Quincke™) spinal needles of 25 g, 26 g, and even 30 g significantly reduced PDPH incidence. These results allowed for the use of spinal anesthesia in age groups and procedures not previously considered suitable.

At the same time, advances in catheter manufacture made possible spinal catheters of 28 g and 32 g which would fit through relatively small spinal needles. Unfortunately, these catheters proved difficult to handle, difficult to make, very expensive, and more ominously, associated with several reports of neurologic damage (i.e., cauda equina syndrome). Many clinicians therefore tried and abandoned them, and they were ultimately removed from the market by the Food and Drug Administration (“FDA”).

A parallel technical development has been the introduction of non-cutting spinal needles, such as the “Pencil Point” type needles, which have been shown to drastically reduce PDPH incidence. Examples of pencil Point type needles include the Sprotte and Whitacre non-cutting spinal needles. In terms of PDPH incidence, a 22 g Sprotte seems to be roughly equivalent to a 25 g or 26 g Quincke, while a 24 g Sprotte or 25 g Whitacre essentially eliminates the risk of PDPH.

The FDA's decision to recall and ban the marketing of microspinal catheters for CSA in the U.S., and its requirement that any new device for CSA be subjected to an extremely stringent pre-market approval process, has resulted in a complete freeze on the development of these products, at least in the United States. Nevertheless, the injection of local anesthetics for the establishment of surgical anesthesia is not the only use to which such devices might profitably be put. In fact, the injection of narcotics, such as FENTANYL®, for analgesia of labor would be a very desirable use of such catheters.

Installing a conventional catheter generally requires various cumbersome steps involving threading long, very thin catheters through a spinal needle. Simply threading a catheter into the end of a spinal needle can be so difficult that some manufacturers include a “threading aid” as part of their kit. Once threaded, a degree of uncertainty exists for the clinician about how far to insert the catheter. Also, a risk exists that a piece of the catheter might be sheared off by the needle if the catheter were to be pulled back during the threading operation. In such case, bits of catheter could potentially be left behind in the intrathecal space. Furthermore, removing the spinal needle while holding the catheter in position can be a challenge. Additionally, attaching a hub/injection adapter to the naked end of the 28 g or 32 g catheter can be even more of a challenge. Finally, once the adapter is successfully attached, the small lumen of the catheter permits only a slow flow of either CSF or anesthetic. In short, the conventional spinal catheter threading operation requires considerable time and effort on the part of a clinician.

One problem of Sprotte and Whitacre non-cutting spinal needles is that the injection orifice is on the side of the needle. Failures of spinal anesthesia have been described as when the needle was “half-in, half-out” of the intrathecal space. Another problem with Sprotte and Whitacre spinal needles is that the smooth curved tip profile provides no definitive feedback signal or “click” when the dura is punctured. Such lack of feedback contributes to uncertainty of catheter tip placement.

Conventional spinal catheters are very long and thin. As such, they are relatively cumbersome to handle without accidental contamination. They also can be difficult to secure to the skin, and can be prone to kinking at the skin or to inadvertent removal by patient movement. This kinking can result in damage to the catheter. Moreover, reports have been made of neurologic damage associated with micro-catheters. Thus, CSA itself has been abandoned in the United States, although it remains popular in Europe.

### DISCLOSURE OF THE INVENTION

In contrast to a conventional spinal catheter, the instant invention provides for simple and straightforward catheter insertion without either threading a catheter through a needle or installing an adapter. The installation procedure is similar to intravenous catheter or single-shot spinal procedures already familiar to clinicians. Placement of the catheter over the inserting needle allows a larger diameter catheter to be inserted. The resulting improved diameter catheter allows easier and faster flow of either CSF or medicating agents.

Insertion of the catheter tip in the intrathecal space with the instant device is more secure. The Pencil Point style non-cutting tip of the support needle promotes a low incidence of PDPH. However, the assembly tip may be shaped to provide a feedback signal when the dura is punctured. Observation of CSF with the instant design further assures a clinician that the entire orifice at the catheter tip is in the intrathecal space.

The chance of neurologic damage is lessened with the shorter catheter of the present invention. The shorter length is less likely to be wedged against a nerve root. More importantly, the larger bore of the improved catheter promotes turbulent flow and improved mixing of any injected fluid will occur with CSF. The improved short catheter, which is inserted to the hub, removes ambiguity about how far to insert. The catheter hub greatly aids fixation to the skin. Contamination during insertion is less likely. Also, kinking at the skin is essentially impossible when a flexible kink sleeve is included.

The ease, simplicity, and relative safety of the improved device may expand the use of continuous spinal anesthesia/analgesia Essentially all lumbar epidurals could be replaced with this apparatus. Similarly, most single-shot spinals maybe replaced with this apparatus “just-in-case” the procedure goes longer than expected, or the level of the block needs adjustment. A number of situations outside the operating environment could benefit from this device, non-exclusively including: acute and chronic pain control with spinal narcotics, labor analgesia, diagnostic taps, and indwelling catheters for continuous peripheral nerve blocks as well as research purposes. In effect, this apparatus can be used in every medical procedure involving needle insertion at the lumbar level of the spine. Versions of the instant device are contemplated to offer improved techniques for the insertion of a wide variety of medical catheters, including arterial lines, major nerve blocks, intraperitoneal catheters, intraventricular (brain) catheters, and intravenous catheters.

The present invention provides an apparatus and method for inserting a spinal catheter in a quick, easy, and straightforward manner. Such a spinal catheter assembly has an outside diameter sized so that withdrawal of the assembly from the subarachnoid space, subsequent to insertion of the assembly thereby, permits the dura mater substantially to reseal a space formerly occupied by the assembly. An assembly typically includes a support needle, a catheter slidably mounted on the support needle, and a central stylet slidably inserted within the support needle. The inserted tip end of a catheter assembly is advantageously configured to produce a feedback signal to indicate dural puncture.

A support needle preferably has a piercing point on a first end and a central hub at a second end. The piercing point protrudes from a front, distal, inserted, or tip, end of a catheter assembly. A piercing point penetrates substantially without cutting, and helps to form a puncture hole through dura mater which automatically may substantially reseal subsequent to retraction of a catheter. A second end of the central stylet generally has a locking hub. The locking hub may carry a first attach structure to connect with corresponding structure of a central stylet.

The front end of the support needle maybe configured cooperatively to form a structural interference with a distal end of a catheter. Such structural interference resists relative motion between the piercing point and the distal end of the catheter during insertion of the catheter into a patient. A rear end of the support needle may carry a support hub having second attach structure to removably connect to the central hub of the central stylet. The first and second attach structures maybe structured to form a removable connection, such as a LUER-LOCK® type connection. The support hub is advantageously made from a transparent material to permit observation of fluid flow therethrough.

A catheter may be characterized as a flexible conduit having distal and proximal ends. Preferred catheters have sufficient transverse flexibility to accommodate patient torso bending movement, whereby substantially to reduce a catheterized patient's awareness of the presence of the catheter. Catheters typically are made from medical grade plastic materials. For example, polyester shrink tube or similar materials maybe used. The distal end of a catheter may be reinforced, in some instances, to resist peel-back from the front end of a support stylet. Such reinforcement may be by way of tip forming or wrappings of fine gauge wire or by a safety ribbon band. The wire or band maybe made from any suitable structurally reinforcing material, including stainless steel. The proximal end of a catheter generally carries a catheter hub having a third attach structure. This third attach structure maybe adapted to structurally interfere in a releasable locking arrangement with a structure carried by the support needle.

The transition from the proximal catheter hub to the catheter body may be reinforced by a kink sleeve segment. The kink sleeve segment maybe constructed of a firm yet flexible material, such as a nylon or other polymer. The kink sleeve is intended to cushion the transition from the hub to the catheter body during bending that will occur after the catheter is inserted and the support needle removed. For example, once the catheter is inserted, the hub may be bent over and taped to the skin, often at an angle of around 90 degrees.

Catheter hubs are typically configured for fluid flow attachment to medical fluid transfer equipment. For example, catheter hubs maybe configured to form LUER-LOCK® type connections with such equipment. It may be further preferred to form the catheter hub for substantially unobtrusive attachment to a patient's skin by way of an intermediary adhesive element or by designing the hub to lay flush against the patient's skin with a connection parallel thereto without a need for bending the catheter.

A spinal catheter assembly maybe installed using a method similar to the following: providing a spinal catheter assembly according to this invention; using conventional spinal needle technique to prepare skin of a patient at an injection site, apply local anesthetic, pierce skin and subcutaneous fascia, and insert a piercing point tip of the catheter assembly; removing the central stylet subsequent to receiving a feedback signal that puncture of the dura mater has occurred; checking for CSF at the support hub; if no CSF is observed, further inserting the assembly until the tip is within the intrathecal space; or if CSF is observed, unlocking the support hub and the catheter hub, and while holding the support needle stationary, advancing the catheter until the catheter hub contacts the skin; removing the support needle and checking for the presence of CSF at the catheter hub; connecting medical fluid transfer apparatus to the catheter hub; and finally, securing the catheter hub to the skin.

These features, advantages, and additional alternative aspects of the present invention will be apparent to those skilled in the art from a consideration of the following detailed description taken in combination with the accompanying drawings.

### BRIEF DESCRIPTION OF THE FIGURES

In the drawings, which illustrate what is currently regarded as the best mode for carrying out the invention and in which like reference numerals refer to like parts in different views or embodiments:

FIG. 1 is an exploded plan assembly view of one embodiment of a catheter assembly according to the present invention.

FIG. 2 is a cross sectional view of a portion of a catheter hub in accordance with one embodiment of the present invention.

FIG. 3 is a plan view of the assembled catheter assembly in FIG. 1.

FIGS. 3A to 3G are plan views of different aspects of interactions between a catheter hub and a support hub, in accordance with the present invention.

FIG. 4 is a cutaway plan view of another embodiment of a catheter hub in accordance with the present invention.

FIG. 5 is a detail view of the distal end portion of the catheter assembly tip, circled and designated by numeral 4, in FIG. 3.

### BEST MODE OF THE INVENTION

The present invention maybe constructed as an integrated spinal needle and catheter assembly 10 (much like an intravenous needle and catheter) in which the catheter 15 is on the outside. Placement of the catheter 15 on the outside provides a number of advantages. First, this design makes insertion significantly easier by eliminating the separate steps of catheter threading, insertion and hub/adapter attachment. A single “stick” is all that is required; once the needle is in, so is the catheter. Since the catheter 15 is larger for a given needle size, its flow and handling characteristics will be much improved, and it is easier and cheaper to manufacture.

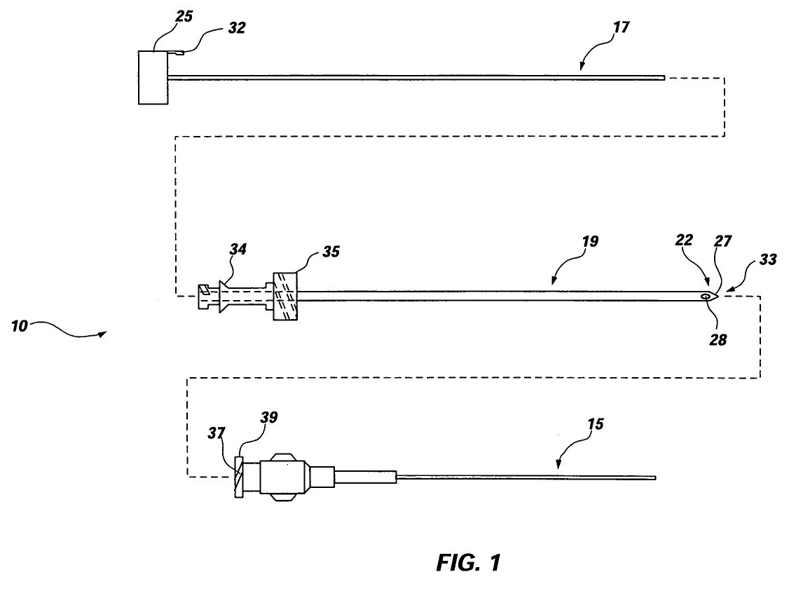

Embodiments of one possible catheter assembly 10, in accordance with the principles of the present invention, are illustrated in FIGS. 1 & 2. The illustrated assembly 10 consists of three components: a central stylet 17, a hollow support needle 19, and a catheter 15. The overall dimensions of the currently preferred embodiment of the assembly 10 are similar to a conventional spinal needle of between 22 g and 24 g.

The innermost component of the assembly is preferably fashioned as a solid central stylet 17. When inserted in the support needle 19 (discussed in detail further herein), the central stylet 17 prevents the entry of extraneous tissue or other material into the support needle opening 28 during insertion. The central stylet may also serve as a “stiffening” portion of the assembly providing extra support and stiffness to the entire assembly. The hub 25 of the central stylet 17 is outermost, or located at an extreme proximal end 26 of assembly 10, because the central stylet 17 is the first to be removed. An attachment structure, such as tab 34, may be located on the hub 25 for retaining the central stylet 17 in the support needle 25. The tab 34 may interact with a corresponding attachment structure on the hub 35 of the support needle 19,

The next layer of the assembly is a removable hollow support needle 19 to support and allow insertion of the catheter 15. This support needle 19 closely resembles a conventional spinal needle. The tip 27 of support needle 19 may have a pencil-point formation to allow penetration of tissue substantially without cutting. As discussed previously herein, this aids in forming a puncture hole through the dura mater which automatically may substantially reseal subsequent to retraction. An opening 28 is located near the tip 27 to allow CSF or other fluids to flow through the support needle 19 from the opening 28 to the hub 35. It will be appreciated that where desired, suitable treatment solutions maybe injected through the support needle 19, to enter a patient's tissue through the opening 28.

The hub 35 of the support needle 19 may beneficially be made of clear plastic to allow visualization of CSF return when the central stylet 17 has been removed. Of course, any present CSF will visibly flow from the distal end 33 of support needle 19 subsequent to removal of the central stylet 17. Optional use of clear plastic or a transparent fluid observation window in the support hub 35 can provide an additional convenience, and minimize loss of CSF.

The central stylet 17 may be attachable to the support needle 19, as illustrated in FIGS. 1 & 2. The central hub 25 typically carries an attach structure, such as tab 32, to interface in a structural interference with an attach structure 34 carried by support hub 35. As illustrated, attach structure 32 and attach structure 34 cooperatively form a slidably engageable joint. Alternative releasable retaining joint configurations, including rotatable attachments such as LUER-LOCK® type joints, may also be used.

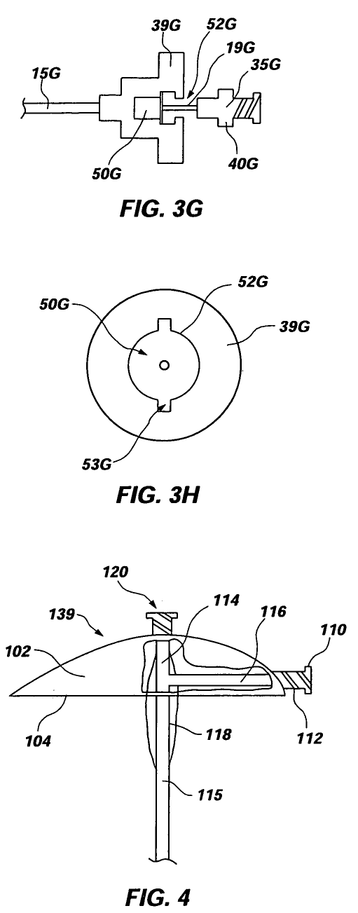

The outermost layer of the assembly 10 is the catheter 15 itself. It preferably is approximately 23 g and about the length of a conventional spinal needle, although different diameters and lengths for use with different procedures is within the scope of the present invention. Conventional plastic catheter material maybe used in its construction. The catheter material maybe reinforced with a flat ribbon internal spring 45 (shown in FIG.5), an internal or external wire wrap, or other reinforcing structure. Alternative materials, and various materials in combination, also maybe used to construct a catheter 15. Suitable catheter material produces a catheter 15 which is fairly stiff and has a sufficiently high tensile strength to maintain structural integrity during insertion, while in the body, and during retraction from a patient. A catheter 15 desirably possesses sufficient transverse flexibility to deform and accommodate patient motion to reduce irritation from the presence of a foreign body.

A slippery nonstick surface is generally provided to ease insertion and removal of the catheter 15. The tip 29 of catheter 15 may be tapered into a curve to blend smoothly into the edge of support needle 19 (see, FIG. 5). The degree of this curved taper may be governed by a tradeoff between the decreased resistance to insertion of an extreme taper versus the fragility and tendency to peelback of a very thin leading edge. A preferred taper provides ease of insertion, a feedback signal to indicate entry of catheter 15 through the dura, and sufficient tensile strength to prevent peelback. The feedback signal may be described as a distinct “click” or a change in required insertion force. The “click” maybe a sonic event, or may be perceptible only through the clinician's fingers in contact with the assembly.

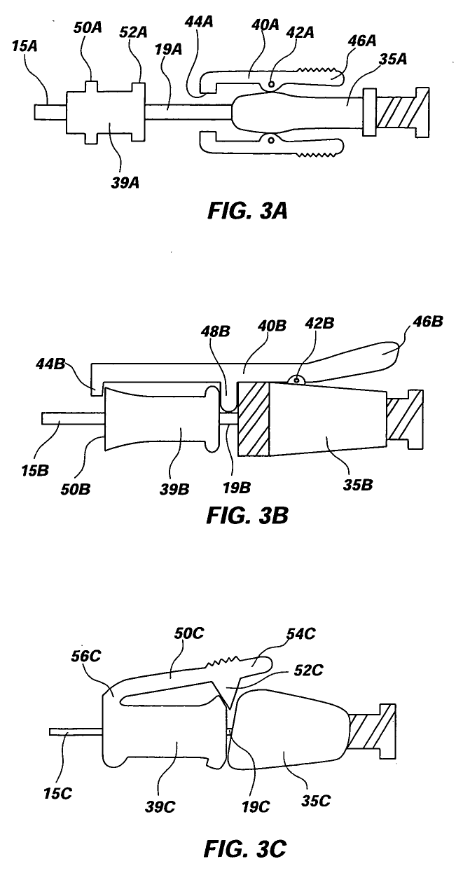

Catheter tips 29 having shapes other than those illustrated in FIGS. 1, 3 & 5 are within contemplation. For example, manufacturing or material requirements may influence the shape of a tip 29. An alternative catheter may include are inforcing wire of fine gauge. Such a wire may be embedded into the material forming the wall of catheter 15 to reinforce against peelback. The wire may also be spiraled along the length of the catheter to provide additional strength to resist collapse, kinking, or breakage of a catheter 15. Alternatively, a flat spring ribbon 45 may be used to provide reinforcement.

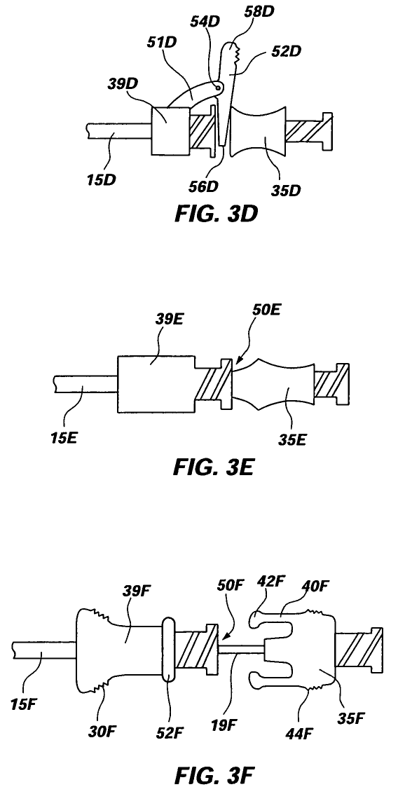

The catheter hub 39 typically includes a LUER-LOCK® type connector, or other attachment structure, for easy and secure connection with common infusion tubing, injection ports, or syringes, and other medical fluid transfer apparatus. Since the catheter 15 may be inserted all the way to the hub 39, a flat, circular flange, or other ergonomically shaped structure, maybe provided on the surface of the hub which rests against the patient's skin to facilitate easy tape fixation. Fixation to the patient's skin maybe accomplished with a slotted circular foam tape. Of course, other tapes or adhesive systems may also be used. A quantity of suitable adhesive or tape could be included in a prepackaged catheter kit. A catheter hub 139 (see FIG. 4) that lays flat against patient's skin and allows attachment of a line at an angle substantially parallel to the skin, rather than generally perpendicular thereto, may be used. Such a catheter hub may be more comfortable for a lengthy procedure.

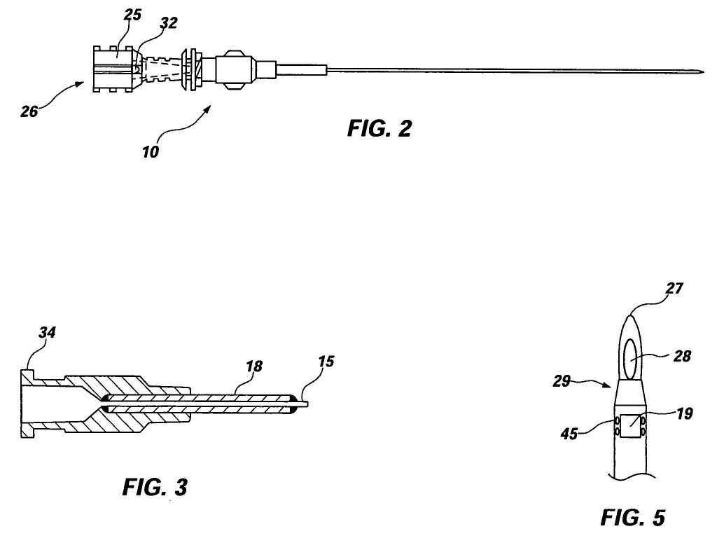

It is desirable to prevent inadvertent premature removal of the support needle 19 from the catheter 15. In the embodiment depicted in FIGS. 1-3, support hub 35 receives thread structure 37 located on the catheter hub 39 and locks with rotation. Such a positive connection may be desirable and can form a LUER-LOCK® or other rotatable-type joint. Other such interlocking or even alternative retaining structure may also be used. For example, a secure friction fit attachment between support needle 19 and catheter 15 is within contemplation in the practice of this invention, as is a structural interference fit of attachment structures similar to shown in connection with tab 32 on the central stylet 17.

FIGS. 3A to 3G depict several aspects of relationships between the hubs of a catheter 15 and a support needle 19 that may prevent premature removal of the support needle 19 or aid in support needle 19 removal at the appropriate point of a procedure. FIG. 3A depicts a support needle 19A having a support hub 35A that includes a plurality of retaining levers 40A. Each lever 40A is attached to the body of the support hub 35A by a pivot structure, such as pin hinge 42A, allowing the distal end of the retaining lever 40A to be rotated away from hub 35A as the proximal end 46A is depressed. The retaining levers 40A expand around the catheter hub 39A allowing the catheter hub 39A and support hub 35A to be slid together as the needle 19A is inserted in the catheter 15A. The distal end of the retaining levers 40A include an attach structure, such as the enlarged end 44A, that interacts with a corresponding attach structure, such as the ridges 50A and 52A on the catheter hub 39A, to retain the hubs in position to one another. Ridges 50A and 52A maybe formed as discrete bumps located on the catheter hub 39A or may be formed as raised ridges running around the entire circumference of the hub 39A. To release support needle 19A from catheter 15A, retaining levers 40A are depressed at the proximal end 46A, which may include a grip structure, and the hubs may then be separated. It will be appreciated that although two retaining levers 40A are depicted, any suitable number maybe used and all such embodiments are within the scope of the present invention.

FIG. 3B depicts a somewhat similar arrangement where a retaining lever 40B is rotatably attached to the support hub 35B and includes an enlarged distal end 44B with a lip that forms a structural interference fit with an attach structure, such as distal end 50B of the catheter hub 39B. Additionally, retaining lever 49B includes a detach assisting structure, such as detach bar 48B that resides between the catheter hub 39B and the support hub 35B in the retained positioned. When proximal end 46B of the retaining lever 40B is depressed distal end 44B rotates out from the catheter hub 39A releasing it. Simultaneously, detach bar 48B presses against the proximal end of the catheter hub 39B, causing support needle 19B to begin withdrawing from catheter 15B. It will be appreciated that although only one retaining lever 40B is shown for clarity, any desired number of retaining levers 40B may be used. Further, although a pin hinge 42B is depicted, any suitable rotatable connection, such as a living hinge formed from injection molded plastic, may be used.

FIG. 3C depicts another detach assisting structure for removing support needle 19C (not shown) from catheter 15C. In such an embodiment, the catheter hub 39C and support hub 35C maybe releasably attached to one another by a friction fit, or by a small amount of a weak adhesive. A detach lever 50C is attached to the catheter hub 39C and includes a detach assisting structure such as detach wedge 52C. Detach lever 50C maybe attached to the catheter hub 39C in any suitable fashion, as by a pin hinge or by forming a living hinge 56C by fashioning detach lever 50C as an extension of the distal end of the catheter hub 39C. The detach wedge 52C maybe disposed so the leading edge thereof is disposed between the distal end of the support hub 35C and the proximal end of the catheter hub 39C body. To actuate detachment, the detaching lever 50C is depressed by pressing on the proximal end 54C thereof causing he detachment wedge 52C to advance further between the hub 35C and 39C forcing them apart and the support needle 19C to withdraw from the catheter 15C.

Another example of aspects of a detach assisting structure is depicted in FIG.3D. A detach lever 52D is rotatably attached catheter hub 39D through a pin hinge 54D or another flexible connection. The attachment may occur on a protrusion, or detachment extension 51D, extending out from the catheter hub 39D body. Detach lever 52D has a detaching end 56D that resides between the catheter hub 39D and the support hub 35D when the hubs are in the retained position. The opposite actuation end 58D of the detach lever 40B may include a grip area formed as roughened surface. Detach lever 52D is actuated by pressing the actuation end 58D in the distal direction causing the detaching end 54D to rotate out from the catheter hub 39D pressing against the distal end of the support hub 35D, causing support needle 19D (not shown) to begin withdrawing from catheter 15D.

FIG. 3E depicts another aspect of a connection between a catheter hub 39E and a support hub 35E. Catheter hub 39E has an enlarged bore opening 50E at its proximal end into which the distal end of support hub 35E maybe inserted upon insertion of the needle 19E (not shown) into catheter 15E. The walls of enlarged bore opening 50E and the distal end of support hub 39E fit together snugly forming a friction fit there between to retain the hubs together. The hubs may be constructed of material selected for a suitable coefficient of friction to maintain the relationship between the hubs.

FIG. 3F depicts a catheter hub 39F including an enlarged bore opening 50F and a support hub 35F having a relationship similar to that described with respect to FIG. 3E. Additional retention structures are also depicted. Support hub 35F includes a lip 40F extending distally from hub body to create a recess 41F. Lip 40F includes an enlarged distal end 42F and may be resilient. As the needle 19F is inserted into catheter 15F and a portion of the support hub 35F is inserted into enlarged bore opening 50F, lip 40F passes over a portion of the catheter hub 39F, flexing outward to allow enlarged end 42F to pass over a ridge 52F on the catheter hub 39F. Enlarged end 42F blockably interacts with ridge 52F to prevent inadvertent removal of the needle 19F. At the appropriate point in the procedure, the hubs may be separated by applying sufficient force to the hubs in opposite directions to cause the lip 40F to flex and allow the enlarged ends 42F to pass over the ridge 52F. Grip points 50F and 44F maybe provided on the catheter hub 39F and support hub 35F, respectively, to assist in the removal of the needle 19F. It will be appreciated that lip 40F maybe formed as an extensions around the entire circumference of the support hub 35F taking the shape thereof, whether generally circular or otherwise, or maybe formed as a plurality of separate extensions, and all such embodiments are within the scope of the present invention.

FIGS. 3G and 3H depict a rotatable retaining relationship between catheter hub 39G and support hub 35G. Support hub 35G includes a number of discrete protrusions, such as retaining tabs 40G at a point along the hub body. Catheter hub 39G includes a enlarged bore opening 50G into which a portion of the body of the support hub 35G my be inserted. The mouth 52G of enlarged bore opening 50G is best depicted in FIG. 3H. A central section of mouth 52G allows the support hub 35G body to pass therethrough, yet is too small to allow the tabs 40G to similarly pass. Mouth 52G includes bays 53G extending into the proximal end of the catheter hub 39G from the central section of the mouth 52G. Each bay 53G corresponds to a tab 40G and allows passage therethrough to the enlarged bore opening.

When support hub 39G is fully inserted into the enlarged bore opening 50G, support needle 19G is fully inserted in the catheter 15G and tabs 40G reside in the enlarged bore opening 50G. Support hub 35G may then be rotated with respect to the catheter hub 39G, so that tabs 40G no longer align with bays 53G. Support needle 19G is then blockably prevented from premature removal from the catheter 15G. Removal can be accomplished by rotating the support hub 39G to align tabs 40G with bays 53G and then slidably withdrawing the support hub 35G. It will be appreciated that although two tabs 40G and bays 53G are depicted, any suitable number may be used.

As best shown in FIG. 3, catheter 15 may include a flexible kink sleeve 18. Kink sleeve covers a portion of the proximal surface of the catheter 15 to protect the area covered against kinking and damage during bending. Desirably, the kink sleeve 18 will begin at the base of the catheter 15 inside the hub 39 (as depicted in FIG. 3) to provide maximum protection, although alternate embodiments where kink sleeve begins distal to the base of the catheter inside the hub 39, or at the base of the hub 39 are within the scope of the present invention. Kink sleeve 18 may extend distally along the length of the catheter 15 to a length appropriate for the planned use of the catheter. Typically, kink sleeve 18 will extend to a length sufficient to prevent kinking of the catheter at the skin of the patient or within the skin and fascia of the patient. Kink sleeve 18 maybe constructed of any suitable flexible material that is medically acceptable, including polymers such as nylon.

When catheter 15 is fully inserted, a portion of the kink sleeve 18 will reside within the skin and fascia of the patient. The hub 39 may then be bent over and taped to the skin, if desired. The kink sleeve 18 acts to protect the catheter 15 during this bending process, which may bend the catheter 15 at an angle of about 90 degrees or more. The kink sleeve 18 absorbs the force of the bend and maintains the catheter 15 in a position allowing flow therethrough. Kinking of the catheter 15 is thus minimized, and maybe prevented. The kink sleeve 18 may be impregnated, coated, or otherwise treated with a biocompatible infection resistant substances to prevent adverse tissue reaction or infection at the catheter entry site. Embodiments where the catheter hub 139 (FIG. 4) lies flat against the skin, allowing attachment at a angle generally perpendicular to insertion may further avoid potential kinking. Similar to kink sleeve 18, the catheter hub 139 may be impregnated, coated, or otherwise treated with a biocompatible infection resistant substances to prevent adverse tissue reaction or infection at the catheter entry site.

FIG. 4 depicts a catheter hub 139 that lies flat against the patient's skin and allows a line to be attached at an angle generally perpendicular to the direction of catheter insertion. The hub 139 includes a body 102 with a generally planar proximal end 104, from which the catheter 115 extends. A kink sleeve 118 may be included on the catheter 115. A bore 114 continuous with the bore of the catheter 115 extends in line therewith through the body 102 to allow placement of the support needle 19 and central stylet 17 through alignment opening 120. A connection bore 116 connects to the bore 114, from an angle generally perpendicular thereto and passes to a connection outlet 110 generally perpendicular to the angle of insertion of catheter 115. The junction between connection bore 116 and bore 114 may be formed as a T-shaped junction as depicted in FIG. 4.

Connection outlet 110 may include a connection structure, such as the LUER-LOCK® type threads 112 depicted in FIG. 4, in order to allow tubing, a connection line, a syringe or other structure to be attached thereto in communication with connection bore 116 and bore 114. A line connected to connection outlet 114 may lay flat on the skin of a patient resulting in a more comfortable connection than a perpendicular connection.

Similarly, alignment opening 120 may include a connection structure, such as LUER-LOCK® type threads, in order to allow tubing, a connection line, a syringe or other structure to be attached thereto in communication with bore 114. Upon withdrawal of the support needle 19 after catheter 15 placement, alignment opening may be closed by capping, with a cap or an injectable port (to provide another point for the introduction of suitable treatment solutions to the catheter 15). In some embodiments, a resealable puncturable membrane may be provided across the alignment opening 120 (or the bore 114 above connection bore 116) to allow insertion of a support needle and central stylet therethrough, while sealing the bore 114 upon their removal.

Catheters 15 may be made from suitable medical grade plastic type materials. For example, polyester shrink tubing may be employed with one embodiment of the device, although it will be appreciated that any suitable material, including other polymers, maybe used. Catheters 15 maybe composed of a single material, or maybe a composite of two or more materials to provide the desired catheter handling characteristics. Fine gauge wire, such as stainless steel wire, or a flat internal ribbon spring 45, maybe incorporated into a catheter wall to improve resistance to peelback. The distal ends may alternatively be reinforced with metal bands. Hubs 25, 35 and 39 are typically also made from medical grade plastic type materials. The central stylet 17 and support needle 19, are typically made from a medically acceptable metal, such as stainless steel or titanium.

The design of this device makes the placement of a spinal catheter 15 quick, easy, and straightforward. It should be so easy, in fact, that most clinicians may choose to use this device for every spinal procedure they perform. The initial steps of skin preparation, local anesthetic infiltration, and needle insertion are identical to those now used with conventional spinal needles. As the assembly 10 is being inserted and the clinician feels the slight “click” upon dural puncture, he or she removes the central stylet 17. If the insertion has been successful, CSF will promptly appear at the hub 35 of the support needle 19. If the dura has not been penetrated, the entire assembly 10 may continue to be advanced until dural puncture is achieved. If desired, the central stylet 17 may be reinserted prior to continued advancement in order to prevent tissue from entering the opening 28.

Once CSF is observed at the hub 35 of the support needle 19, the clinician can be certain that the tip 29 of the catheter 15 is within the intrathecal space. If desirable for the procedure, the clinician may continue to advance the hollow stylet/catheter 19/15 assembly another centimeter or so. At this point, the hub 35 of the hollow stylet 19 is typically twisted to unlock it from the catheter hub 39 or 139, and while holding the hollow stylet 19 stationary, the catheter 15 is advanced all the way until the hub 39 or 139 contacts the patient's skin. For embodiments including a kink sleeve 18, this advancement inserts, or further inserts, the kink sleeve 18 within the patient's skin.

At this point, the hollow support needle 19 may be removed, and the appearance of CSF at the catheter hub 39 or 139 will confirm the correct placement of the catheter 15. The desired injection port, tubing, or other medical fluid transfer apparatus, may then be attached to the catheter hub 39 (or 139) such as by way of attach structure 37 (or 112). Where necessary, the catheter 15 may be bent and taped to the patient's skin before of after the attachment of the corresponding apparatus, if required. Where included, kink sleeve 18 protects the catheter 15 from kinking and damage at the bend. A piece of slotted, circular foam tape (which might also be treated with an antimicrobial) may also be applied to fix the hub 39 or 139 to the skin, prevent dislodging of the catheter 15, and cushion the patient to reduce potential irritation from the hub 39 or 139.

The catheter 15 may then be left in place for as long as clinically necessary and, assuming adequate tensile strength, be easily and safely removed when appropriate. At the time of removal, since the non-cutting point 22 of the support needle 19 never lacerated any fibers in the dural membrane, the mesh-like fibers may relax to their original position, thus automatically closing the dural puncture. Therefore the PDPH incidence is expected to be in agreement with Sprotte and Whitacre needles, despite the luxury of a reasonably large catheter 15 in a device according to the instant invention.

The present invention maybe embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A spinal catheter assembly having an outside diameter sized so that withdrawal of the spinal catheter assembly from dura mater of a spine, subsequent to insertion of said assembly therethrough, permits the dura mater substantially to reseal a space formerly occupied by the assembly, said spinal catheter assembly comprising:
a support needle having a first end defining a non-cutting piercing point, said support needle further comprising a hollow bore with an opening proximate said first end allowing access to said bore; and
a spinal catheter slidably mounted on a portion of said support needle such that said first end of said support needle protrudes from said spinal catheter exposing said non-cutting piercing point and said opening.

2. The spinal catheter assembly of claim 1, wherein said non-cutting piercing point comprises a pencil-point tip.

3. The spinal catheter assembly of claim 1, wherein said catheter assembly has a tip configured and arranged to provide a feedback signal to indicate dural puncture.

4. The spinal catheter assembly of claim 1, wherein:
a rear end of said support needle carries a support hub having a first attach structure; and
a proximal end of said catheter carries a catheter hub having a second attach structure configured to removably attach to the first attach structure carried by said support hub.

5. The spinal catheter assembly of claim 4, wherein the first and second attach structures comprise a LUER-LOCK® type connection.

6. The spinal catheter assembly of claim 4, wherein said catheter hub is configured for substantially unobtrusive attachment to a patient's skin by way of an intermediary adhesive element.

7. The spinal catheter assembly of claim 4, wherein said catheter hub is configured for attachment to medical fluid transfer equipment by an attachment structure to form a connection generally perpendicular to a direction of catheter insertion.

8. The spinal catheter assembly of claim 1, wherein:
a rear end of said support needle carries a support hub; and
a proximal end of said catheter carries a catheter hub having a detach structure configured to detach the catheter hub from the support hub.

9. The spinal catheter assembly of claim 1, wherein:
a proximal end of said catheter carries a catheter hub; and
a rear end of said support needle carries a support hub having a detach structure configured to detach the catheter hub from the support hub.

10. The spinal catheter assembly of claim 1, wherein said catheter comprises a conduit formed from a first material and radially reinforced at a distal end by a second material.

11. The spinal catheter assembly of claim 10, wherein said second material is selected from the group comprising a stainless steel wire and a ribbon spring.

12. The spinal catheter assembly of claim 1, wherein said catheter comprises a force absorbing structure to prevent kinking when the catheter is bent.

13. The spinal catheter assembly of claim 12, wherein said force absorbing structure comprises a ribbon spring.

14. The spinal catheter assembly of claim 12, wherein said force absorbing structure comprises a kink sleeve disposed on a portion thereof.

15. The spinal catheter assembly of claim 1, further comprising a central stylet slidably mounted in said support needle to prevent the entry of matter through said opening proximate said first end.

16. A catheter assembly for inserting a distal end of a spinal catheter through dura mater into a spine of a patient, said catheter assembly comprising:
a support needle having a proximal end and a non-cutting piercing point at a distal end, said support needle configured to resist relative motion between said distal end of said catheter and said non-cutting piercing point during insertion of said catheter assembly into the patient;
wherein said catheter is carried exterior to said support needle to expose said non-cutting piercing point when positioned for said inserting.

17. The catheter assembly of claim 16, wherein said catheter has an exterior diameter such that withdrawal of said catheter from said dura mater, subsequent to insertion of the catheter assembly therethrough, permits said dura mater substantially to reseal a space formerly occupied by said catheter.

18. The catheter assembly of claim 17, wherein:
said proximal end of said support needle carries a support hub having a first attach structure;
a proximal end of said catheter carries a catheter hub having a second attach structure configured to interface in removable interference with said first structure carried by said support hub.

19. The catheter assembly of claim 16, wherein said catheter further comprises a radially reinforcing material located at a distal end of said catheter, said reinforcing material resisting peel-back of said catheter from said support needle.

20. The catheter assembly of claim 16, having a distal end of said assembly being constructed to provide a perceptible feedback signal when said distal end of said catheter penetrates said dura mater.

21. The catheter assembly of claim 16, characterized in said catheter hub further being configured for attachment to medical fluid transfer equipment having structure to form a LUER-LOCK® type connection.

22. The catheter assembly of claim 16, wherein a catheter hub is configured for attachment to medical fluid transfer equipment by an attachment structure to form a connection generally perpendicular to a direction of catheter insertion.

23. The catheter assembly of claim 16, wherein said catheter comprises a kink sleeve disposed on a portion thereof, said kink sleeve configured to prevent kinking of said catheter when said catheter is bent during use.

24. A method for installing a spinal catheter assembly, said method comprising:
providing a spinal catheter assembly having:
a support needle with a piercing point at a distal end, said support needle having a proximal end with a support hub and an opening to allow flow from a point near the distal end thereof to said proximal end;
a catheter, having a proximal end with a catheter hub, slidably mounted on said support needle to expose said piercing point, said catheter having an outside diameter sufficiently small so that withdrawal of said catheter from dura mater, subsequent to insertion of said catheter assembly therethrough, permits said dura mater substantially to reseal a space formerly occupied by said catheter; wherein
said catheter hub and said support hub are configured to form a locking interference therebetween; and
a central stylet slidably mounted in said support needle to prevent the entry of matter through said opening;
using a conventional spinal needle technique to prepare skin of a patient at an injection site, apply local anesthetic, and insert a tip of said catheter assembly, said tip comprising said piercing point;
removing said central stylet subsequent to receiving a feedback signal that puncture of said dura mater has occurred;
checking for cerebrospinal fluid at said support hub;
if no cerebrospinal fluid is observed, replacing said central stylet and further inserting said assembly until said tip is an intrathecal space;
once cerebrospinal is observed, unlocking said support hub and said catheter hub, and while holding said support needle stationary, advancing said catheter until said catheter hub contacts the skin;
removing said support needle and checking for the presence of CSF at said catheter hub;
connecting medical fluid transfer apparatus to said catheter hub; and
securing said catheter hub to the skin.

25. A spinal catheter comprising:
a catheter body comprising an elongated hollow tube, said catheter body configured to be slidably mounted on an exterior of a support needle;
a kink sleeve disposed on a portion of said catheter body, said kink sleeve configured to prevent kinking of said catheter body, when said catheter body is bent during use.

26. A spinal catheter comprising:
a catheter body comprising an elongated hollow tube, said catheter body configured to be slidably mounted on an exterior of a support needle;
a catheter hub configured for attachment to medical fluid transfer equipment by an attachment structure to form a connection generally perpendicular to a longitudinal axis of said catheter body.
